(19) United States
(12) Patent Application Publication
(10) Pub. No.: US 2024/0057655 A1
(43) Pub. Date: Feb. 22, 2024

(54) COMPOSITIONS FOR COATING EDIBLE SUBSTRATES AND METHODS OF MAKING AND USING THE SAME

(71) Applicant: Sensient Colors LLC, St. Louis, MO (US)

(72) Inventor: Beverly A. Schad, Union, MO (US)

(21) Appl. No.: 18/339,800

(22) Filed: Jun. 22, 2023

Related U.S. Application Data
(63) Continuation of application No. 16/970,175, filed on Aug. 14, 2020, now abandoned.
(60) Provisional application No. 62/632,362, filed on Feb. 19, 2018.

(51) Int. Cl. A23P 20/10; A61K 9/20; A61K 9/28

(52) U.S. Cl. CPC A23P 20/105; A61K 9/2018; A61K 9/2866

(57) ABSTRACT

A composition for coating an edible substrate including about 85.0 wt % to about 95.0 wt % of a cellulose film former (e.g., hydroxypropyl methyl cellulose), about 5.0 wt % to about 15.0 wt % of a plasticizer (e.g., glycerin), and about 1.0 wt % to about 4.0 wt % of a lubricant selected from sunflower oil, vegetable oil, hydrogenated vegetable oil, mineral oil, a fatty acid, or a combination thereof, relative to the total weight of non-water components of the composition. The composition may be applied to a nutritional supplement, a pharmaceutical, a tablet, a capsule, a softgel, a granule, a microparticle, a nanoparticle, a seed, a sugar sphere, gum chew base, chewable gel, or a gummy substrate

### CROSS-REFERENCE TO RELATED APPLICATIONS

This application is a continuation of U.S. patent application Ser. No. 16/970,175, filed Aug. 14, 2020, which is the U.S. national stage entry, under 35 U.S.C. § 371, of International Application Number PCT/US2019/018613, filed Feb. 19, 2019, which claims priority to U.S. Provisional Application No. 62/632,362, filed Feb. 19, 2018, the entire contents of each of which are hereby incorporated by reference.

### FIELD OF USE

The present disclosure relates to compositions for coating edible substrates and methods for making and using the same, for use in the food, pharmaceutical, and nutraceutical industries.

### INTRODUCTION

There is a need for a commercially viable cellulose-based composition to provide hydrophilic edible films that have good barrier properties.

### SUMMARY

In one aspect, a composition is provided for coating an edible substrate including about 85.0 wt % to about 95.0 wt % of a cellulose film former, about 5.0 wt % to about 15.0 wt % of a plasticizer, and about 1.0 wt % to about 4.0 wt % of a lubricant selected from sunflower oil, vegetable oil, hydrogenated vegetable oil, mineral oil, a fatty acid, or a combination thereof, relative to the total weight of non-water components of the composition.

In another aspect, a method is provided of forming an edible coating on an edible substrate including spraying the composition onto the substrate.

In another aspect, nutritional supplement, a pharmaceutical, a tablet, a capsule, a softgel, a granule, a microparticle, a nanoparticle, a seed, a sugar sphere, gum chew base, chewable gel, or a gummy substrate is provided including an edible substrate and the composition thereon.

### BRIEF DESCRIPTION OF THE FIGURES

FIG. 1 shows (top image) a photograph of coating solutions including (from left to right) 0%, 1%, 2%, 3%, and 4% oil by weight of the non-water components of the coating composition, and (bottom image) a photograph of upper coated tablets depicting coatings according to the present disclosure including (from left to right) 0%, 1%, 2%, 3%, and 4% oil by weight of the non-water components of the coating composition.

FIG. 2 shows a graph depicting a slip analysis of edible coatings according to the present disclosure. The smaller (i.e., more negative/more diagonal) the downward slope of a line, the better the slip for that sample. The larger (i.e., less negative/more horizontal) the downward slope of a line, the more drag or the more non-slip the sample surface is.

### DETAILED DESCRIPTION

Before any embodiments of the disclosure are explained in detail, it is to be understood that the disclosure is not limited in its application to the details of construction and the arrangement of components set forth in the following description. The disclosed invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. The present disclosure also contemplates other embodiments “comprising,” “consisting of” and “consisting essentially of,” the embodiments or elements presented herein, whether explicitly set forth or not.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application. It also is understood that for the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” is used synonymously with the term “approximately.” Illustratively, the use of the term “about” indicates that a recited value may include additional values slightly outside the recited values. This variation may be due to conditions such as experimental error, manufacturing tolerances, variations in equilibrium conditions, and the like. In some embodiments, the term “about” may include the cited value plus or minus 2.5%, 5%, 7.5%, or 10%, among others. It is to be understood that when the term “about” is used to modify a value, the value being modified is also expressly included. For example, “at least about 5” expressly includes at least 5, in addition to at least about 5. Similarly, by way of example, “at most about 95” expressly includes at most 95, in addition to at most about 95.

The term “substantially free”, as used herein unless otherwise defined, means that a composition comprises less than about 5%, less than about 4%, less than about 3%, less than about 2%, less than about 1%, less than about 0.9%, less than about 0.8%, less than about 0.7%, less than about 0.6%, less than about 0.5%, less than about 0.4%, less than about 0.3%, less than about 0.2%, less than about 0.1%, less than about 0.05% or less than about 0.01% by weight of, or none of the indicated component.

Hydroxypropylmethyl cellulose (HPMC) is used as a film former and in some cases as an emulsifier, protective colloid, and suspending agent. The degree of substitution and chain length of IPMC may be used to improve various barrier properties. Moreover, the water vapor permeability (WVP) of composite films with IPMC plus a plasticizer with oil present can afford WVP values as low as 1.90×10⁻¹⁰ (g m⁻¹ s⁻¹ Pa⁻¹), in which glycerin is the main plasticizer.

Disclosed herein are edible coating compositions comprising at least one of hydroxypropylmethyl cellulose (HPMC), a plasticizer (e.g., glycerin), an oil (e.g., sunflower oil, vegetable oil), water, and combinations thereof. In one embodiment, the edible coating composition comprises or consists essentially of HPMC, glycerin, and sunflower oil. The edible coating composition may comprise or consist essentially of HPMC, a plasticizer, an oil, and optionally water. The edible coating composition may comprise or consist essentially of HPMC, glycerin, sunflower oil, and optionally water. Also disclosed are methods of making edible coating compositions.

Further disclosed are methods of using edible coating compositions, including methods of making edible coatings. The edible coatings comprise at least one of hydroxypropyl methylcellulose (HPMC), a plasticizer (e.g., glycerin), an oil (e.g., sunflower oil, vegetable oil), water, and combinations thereof. In one embodiment, the edible coating comprises or consists essentially of HPMC, glycerin, and sunflower oil. The edible coating may comprise or consist essentially of HPMC, a plasticizer, an oil, and optionally water. The edible coating may comprise or consist essentially of HPMC, glycerin, sunflower oil, and optionally water.

The edible coating composition can form an edible coating that may have improved properties when compared to conventional coatings that contain HPMC. In particular, the coating may have at least one of improved slip attributes, improved shine, improved stability, improved spray delivery, higher spray efficiency, and combinations thereof compared to conventional HPMC/glycerin coatings with or without oil.

### 1. Edible Coating Compositions

The edible coating compositions of the present disclosure may comprise at least one of a cellulose film former (e.g., hydroxypropyl methylcellulose (HPMC)), a plasticizer (e.g., glycerin), a lubricant (e.g., sunflower oil), water, colorants, and combinations thereof. The composition is useful for coating an edible substrate. The composition is typically shipped in dry form and reconstituted in an aqueous media. The edible coating may have at least one of improved slip attributes, improved stability, improved shine, improved spray delivery, higher spray efficiency, and combinations thereof.

a. Cellulose Film Former

A cellulose film former may include a molecule comprising at least one cellulose polymer or derivative modified with small amounts of propylene glycol ether groups attached to the cellulose anhydroglucose chain affording binding properties that enhance the reinforcing film properties of film applications. Cellulose film formers may provide smooth, clear, film-forming coating suspensions and enhance rheological mechanical strength properties of dry film coating gel matrices. Examples of cellulose film formers include, but are not limited to, hydroxypropyl methyl cellulose (HPMC), carboxymethyl cellulose (CMC), hydroxypropyl cellulose (HPC), methylcellulose (MC), hydroxyethyl cellulose (HEC), methylethyl cellulose (MEC), or salts thereof and the like.

HPMC may be used to form the edible coating compositions. Examples of commercially-available HPMC may include, for example, Spectracel™ (available commercially from Sensient Technologies, Inc., St. Louis, MO), Mantrocel™ (available commercially from Sensient Technologies, Inc., St. Louis, MO), Benecel™ (available commercially from Ashland, Inc., Covington, KY), Methocel™ (available commercially from DOW Chemical Company, Midland, MI), among others.

In some embodiments, the amount of HPMC (by weight of the non-water components) in the composition may be at least about 85.0%, at least about 86.0%, at least about 87.0%, at least about 88.0%, at least about 89.0%, at least about 90.0%, at least about 91.0%, at least about 92.0%, at least about 93.0%, at least about 94.0%, or at least about 95.0%.

Further, the amount of HPMC (by weight of the non-water components) in the composition may be at most about 95.0%, at most about 94.0%, at most about 93.0%, at most about 92.0%, at most about 91.0%, at most about 90.0%, at most about 89.0%, at most about 88.0%, at most about 87.0%, at most about 86.0%, or at most about 85.0%.

This includes embodiments in which the HPMC may be present in the composition in amounts (by weight of the non-water components) ranging from about 85.0% to about 95.0%, preferably from about 87.0% to about 93.0%.

The HPMC may have a viscosity in 2% aqueous solution at 20° C. of at least about 1.0 cP, at least about 2.0 cP, at least about 3.0 cP, at least about 4.0 cP, at least about 5.0 cP, at least about 6.0 cP, at least about 7.0 cP, at least about 8.0 cP, at least about 9.0 cP, at least about 10.0 cP, at least about 11.0 cP, at least about 12.0 cP, at least about 13.0 cP, at least about 14.0 cP, or at least about 15.0 cP. The HPMC may have a viscosity in 2% aqueous solution at 20° C. of at most about 30.0 cP, at most about 29.0 cP, at most about 28.0 cP, at most about 27.0 cP, at most about 26.0 cP, at most about 25.0 cP, at most about 24.0 cP, at most about 23.0 cP, at most about 22.0 cP, at most about 21.0 cP, at most about 20.0 cP, at most about 19.0 cP, at most about 18.0 cP, at most about 17.0 cP, at most about 16.0 cP, at most about 15.0 cP, at most about 14.0 cP, at most about 13.0 cP, at most about 12.0 cP, at most about 11.0 cP, at most about 10.0 cP, at most about 9.0 cP, at most about 8.0 cP, at most about 7.0 cP, at most about 6.0 cP, or at most about 5.0 cP. This includes embodiments in which the IPMC may have a viscosity in 2% aqueous solution at 20° C. ranging from about 1.0 cP to about 30.0 cP, such as from about 3.0 cP to about 30.0 cP, from about 3.0 cP to about 15.0 cP, or from about 6.0 cP to about 15.0 cP. In embodiments, the viscosity can vary based on stirring rate and air entrapments in solution.

b. Plasticizers

Plasticizers used in edible products may be used to form the edible coating composition. Plasticizers may enhance the film characteristics of the coating, such as adhesion, flexibility, permeability, and the like. In embodiments, plasticizers may include medium chain triglycerides, fatty acids, fatty acid derivatives, and combinations thereof. Examples of plasticizers include, without limitation, at least one of glycerin, sorbitol, propylene glycol dicaprylate/dicaprate, medium chain triglycerides (e.g., fractionated coconut oil), glyceryl monostearate, propylene glycol, polypropylene glycol, polyethylene glycol, triacetin, dibutyl sebacate, triglycerides, acetylated monoglycerides, glycerol monostearates, glycerin monostearate, oleic acid, stearic acid, tributyl citrate, acetyltributyl citrate, dibutyl phthalate, triethyl citrate, triethanolamine, aqueous emulsions of glyceryl monostearate and triethyl citrate, and combinations thereof. Particularly suitable plasticizers include glycerin.

In some embodiments, the amount of plasticizer (by weight of the non-water components) in the composition may be at least about 5.0%, at least about 5.1%, at least about 5.2%, at least about 5.3%, at least about 5.4%, at least about 5.5%, at least about 5.6%, at least about 5.7%, at least about 5.8%, at least about 5.9%, at least about 6.0%, at least about 6.1%, at least about 6.2%, at least about 6.3%, at least about 6.4%, at least about 6.5%, at least about 6.6%, at least about 6.7%, at least about 6.8%, at least about 6.9%, at least about 7.0%, at least about 7.1%, at least about 7.2%, at least about 7.3%, at least about 7.4%, at least about 7.5%, at least about 7.6%, at least about 7.7%, at least about 7.8%, at least about 7.9%, at least about 8.0%, at least about 8.1%, at least about 8.2%, at least about 8.3%, at least about 8.4%, at least about 8.5%, at least about 8.6%, at least about 8.7%, at least about 8.8%, at least about 8.9%, at least about 9.0%, at least about 9.1%, at least about 9.2%, at least about 9.3%, at least about 9.4%, at least about 9.5%, at least about 9.6%, at least about 9.7%, at least about 9.8%, at least about 9.9%, at least about 10.0%, at least about 10.1%, at least about 10.2%, at least about 10.3%, at least about 10.4%, at least about 10.5%, at least about 10.6%, at least about 10.7%, at least about 10.8%, at least about 10.9%, at least about 11.0%, at least about 11.1%, at least about 11.2%, at least about 11.3%, at least about 11.4%, at least about 11.5%, at least about 11.6%, at least about 11.7%, at least about 11.8%, at least about 11.9%, at least about 12.0%, at least about 12.1%, at least about 12.2%, at least about 12.3%, at least about 12.4%, at least about 12.5%, at least about 12.6%, at least about 12.7%, at least about 12.8%, at least about 12.9%, at least about 13.0%, at least about 13.1%, at least about 13.2%, at least about 13.3%, at least about 13.4%, at least about 13.5%, at least about 13.6%, at least about 13.7%, at least about 13.8%, at least about 13.9%, at least about 14.0%, at least about 14.1%, at least about 14.2%, at least about 14.3%, at least about 14.4%, at least about 14.5%, at least about 14.6%, at least about 14.7%, at least about 14.8%, at least about 14.9%, or at least about 15.0%.

Further, the amount of plasticizer (by weight of the non-water components) in the composition may be at most about 15.0%, at most about 14.9%, at most about 14.8%, at most about 14.7%, at most about 14.6%, at most about 14.5%, at most about 14.4%, at most about 14.3%, at most about 14.2%, at most about 14.1%, at most about 14.0%, at most about 13.9%, at most about 13.8%, at most about 13.7%, at most about 13.6%, at most about 13.5%, at most about 13.4%, at most about 13.3%, at most about 13.2%, at most about 13.1%, at most about 13.0%, at most about 12.9%, at most about 12.8%, at most about 12.7%, at most about 12.6%, at most about 12.5%, at most about 12.4%, at most about 12.3%, at most about 12.2%, at most about 12.1%, at most about 12.0%, at most about 11.9%, at most about 11.8%, at most about 11.7%, at most about 11.6%, at most about 11.5%, at most about 11.4%, at most about 11.3%, at most about 11.2%, at most about 11.1%, at most about 11.0%, at most about 10.9%, at most about 10.8%, at most about 10.7%, at most about 10.6%, at most about 10.5%, at most about 10.4%, at most about 10.3%, at most about 10.2%, at most about 10.1%, at most about 10.0%, at most about 9.9%, at most about 9.8%, at most about 9.7%, at most about 9.6%, at most about 9.5%, at most about 9.4%, at most about 9.3%, at most about 9.2%, at most about 9.1%, at most about 9.0%, at most about 8.9%, at most about 8.8%, at most about 8.7%, at most about 8.6%, at most about 8.5%, at most about 8.4%, at most about 8.3%, at most about 8.2%, at most about 8.1%, at most about 8.0%, at most about 7.9%, at most about 7.8%, at most about 7.7%, at most about 7.6%, at most about 7.5%, at most about 7.4%, at most about 7.3%, at most about 7.2%, at most about 7.1%, at most about 7.0%, at most about 6.9%, at most about 6.8%, at most about 6.7%, at most about 6.6%, at most about 6.5%, at most about 6.4%, at most about 6.3%, at most about 6.2%, at most about 6.1%, at most about 6.0%, at most about 5.9%, at most about 5.8%, at most about 5.7%, at most about 5.6%, at most about 5.5%, at most about 5.4%, at most about 5.3%, at most about 5.2%, at most about 5.1%, or at most about 5.0%.

This includes embodiments in which the plasticizer may be present in the composition in amounts (by weight of the non-water components) ranging from about 5.0% to about 15.0%, preferably from about 6.0% to about 10.0%.

c. Lubricants

Lubricants used in edible products may be used to form the edible coating composition. A lubricant may enhance esophageal transit ease, film slipperiness, and surface flowability. Examples of lubricants include, without limitation, at least one of sunflower oil, vegetable oil, hydrogenated vegetable oil, mineral oil, lipid-type fatty acids, and combinations thereof. In embodiments, the lubricant may be amphoteric. Alternately, the lubricant may be non-amphoteric. In embodiments, an oil may be neutral or nonpolar. Particularly suitable lubricants include sunflower oil, other lipid-type fatty acids, and combinations thereof. Alternately, lubricants and/or oils may be omitted from the edible coating composition altogether, particularly if the edible substrate does not need absolute moisture protection.

In some embodiments, the amount of lubricant (by weight of the non-water components) in the composition may be at least about 1.0%, at least about 1.1%, at least about 1.2%, at least about 1.3%, at least about 1.4%, at least about 1.5%, at least about 1.6%, at least about 1.7%, at least about 1.8%, at least about 1.9%, at least about 2.0%, at least about 2.1%, at least about 2.2%, at least about 2.3%, at least about 2.4%, at least about 2.5%, at least about 2.6%, at least about 2.7%, at least about 2.8%, at least about 2.9%, at least about 3.0%, at least about 3.1%, at least about 3.2%, at least about 3.3%, at least about 3.4%, at least about 3.5%, at least about 3.6%, at least about 3.7%, at least about 3.8%, at least about 3.9%, or at least about 4.0%.

In some embodiments, the amount of lubricant (by weight of the non-water components) in the composition may be at most about 4.0%, at most about 3.9%, at most about 3.8%, at most about 3.7%, at most about 3.6%, at most about 3.5%, at most about 3.4%, at most about 3.3%, at most about 3.2%, at most about 3.1%, at most about 3.0%, at most about 2.9%, at most about 2.8%, at most about 2.7%, at most about 2.6%, at most about 2.5%, at most about 2.4%, at most about 2.3%, at most about 2.2%, at most about 2.1%, at most about 2.0%, at most about 1.9%, at most about 1.8%, at most about 1.7%, at most about 1.6%, at most about 1.5%, at most about 1.4%, at most about 1.3%, at most about 1.2%, at most about 1.1%, or at most about 1.0%.

This includes embodiments in which the lubricant may be present in the composition in an amount (by weight of the non-water components) ranging from about 1.0% to about 4.0%, preferably from about 2.0% to about 3.0%.

d. Plasticizer:Lubricant Ratio

In some embodiments, the ratio of amount of plasticizer to amount of oil (by weight) in the composition may be at least 0.02, 0.2, 0.4, 0.6, 0.8, 1.0, 1.2, 1.25, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, or 15.0.

In some embodiments, the ratio of amount of plasticizer to amount of lubricant (by weight) in the composition may be at most 15.0, 14.9, 14.8, 14.7, 14.6, 14.5, 14.4, 14.3, 14.2, 14.1, 14.0, 13.9, 13.8, 13.7, 13.6, 13.5, 13.4, 13.3, 13.2, 13.1, 13.0, 12.9, 12.8, 12.7, 12.6, 12.5, 12.4, 12.3, 12.2, 12.1, 12.0, 11.9, 11.8, 11.7, 11.6, 11.5, 11.4, 11.3, 11.2, 11.1, 11.0, 10.9, 10.8, 10.7, 10.6, 10.5, 10.4, 10.3, 10.2, 10.1, 10.0, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.25, 1.2, 1.0, 0.8, 0.6, 0.4, 0.2, or 0.02.

In some embodiments, the ratio of amount of plasticizer to amount of lubricant (by weight) in the composition may be about 1.0, about 1.25, about 1.5, about 1.75, about 2.0, about 3.0, about 4.0, about 5.0, about 6.0, about 7.0, about 8.0, about 9.0, about 10.0, about 11.0, about 12.0, about 13.0, about 14.0, or about 15.0.

This includes embodiments in which the ratio of the amount of plasticizer to amount of lubricant may be present in the composition (by weight) ranging from about 1.25 to about 15.0.

e. Water

The edible coating compositions disclosed herein may optionally contain water. In some embodiments containing water, the amount of water (by weight of the entire composition) in the composition may be at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

Further, in some embodiments containing water, the amount of water (by weight of the entire composition) in the composition may be at most about 99.9%, at most about 99%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, or at most about 5%.

This includes embodiments in which the water may be present in the composition in amount (by weight of the entire composition) ranging from about 1% to about 99.9%, such as from about 5% to about 95%, or from about 50% to about 99%. In some embodiments, the balance of the edible coating composition may be water.

f. Additional Composition Additives

The edible coating compositions disclosed herein may also comprise additional additives known to those of skill in the art. Examples of suitable additives include, without limitation, colorants (synthetic, natural exempt, and natural non-exempt), opacifying agents, flavorings, sweeteners, taste maskants, processing aids, slip aids, detackifiers, lubricants, surfactants, buffering agents, solvents, sodium bicarbonate, sodium stearate, and the like. In some embodiments, additional additives do not affect at least one of the slip attributes, shine, stability, spray delivery, spray efficiency, moisture uptake, or clarity of the edible coatings.

Examples of colorants include dyes, lakes, and pigments and may include, but are not limited to, titanium dioxide, iron oxides, dyes such as, for example, FD&C Lakes, Carmine Lake, FD&C Blue no. 1, FD&C Blue no. 2, FD&C Red no. 3, FD&C Red no. 40, FD&C Yellow no. 5, FD&C Yellow no. 6, FD&C Green no. 3, alumina, talc, annatto extract, calcium carbonate, canthaxanthin, caramel, β-carotene, carmine, dihydroxyacetone, turmeric oleoresin, cochineal extract, gardenia yellow, gardenia blue, beet powder, grape skin extract, riboflavin, purple sweet potato, red sweet potato, chlorophyll-containing extracts, purple blend (available from Sensient Colors, Inc., No. 53219), anthocyanins, carotenoids, Pure-S, SupraRed, Natural Blues, Caramel replacements, carmine high tint, pearlescent pigments, SensiPearl™, Intense Silver, and Bright Silver (available from Sensient Colors, Inc.), natural colorants, and the like. Other examples of colorants are found in 21 C.F.R. §§ 73 and 74, which are hereby fully incorporated by reference.

Examples of opacifying agents include titanium dioxide, clays, and divalent salts such as zinc oxides, dicalcium phosphate, dicalcium phosphate dehydrate, tricalcium phosphate, calcium carbonate, precipitated calcium carbonate, and Avalanche (available from Sensient Colors, Inc.). In certain embodiments, no opacifying agents are included in the edible coating composition or the edible coating.

Examples of flavorings may be synthetic or artificial flavorings, natural flavorings, or any mixture thereof and may include, but are not limited to, sensates, flavonoids, antioxidants, natural flavorants, synthetic flavorants, bioflavonoids, flavones, flavone, flavonol, flavanonol, isoflavones, ethyl vanillin, tangerine flavor, lemon flavor, lemon extract, liquid caramel, spearmint oil, orange flavor, almond, amaretto, apple, green apple, apple-cherry-berry, apple-honey, apricot, bacon, balls of fire, banana, barbeque, beef, roast beef, beef steak, berry, berry blue, birch beer/spruce beer, blackberry, bloody mary, blueberry, boysenberry, brandy, bubble gum, butter, butter pecan, buttermilk, butterscotch, candy corn, cantaloupe, cantaloupe lime, caramel, carrot, cassia, caviar, celery, cereal, champagne, cherry, cherry cola, cherry maraschino, wild cherry, black cherry, red cherry, cherry-cola, chicken, chocolate, chocolate almond, cinnamon spice, citrus, citrus blend, citrus-strawberry, clam, cocoa, coconut, toasted coconut, coffee, coffee almond, cola, cola-vanilla, cookies & cream, cool, cotton candy, cranberry, cranberry-raspberry, cream, cream soda, dairy type cream, creme de menthe, cucumber, black currant, dulce de leche, egg nog, pork fat, type fat, anchovy fish, herring fish, sardine fish, frankfurter, fiery hot, fried garlic, sauteed garlic, gin, ginger ale, ginger beer, graham cracker type, grape, grape grapefruit, grapefruit-lemon, grapefruit-lime, grenadine, grill, guarana, guava, hazelnut, honey, hot, roasted honey, ice cream cone, jalapeno, key lime, kiwi, kiwi-banana, kiwi-lemon-lime, kiwi-strawberry, kola champagne, lard type, lemon, lemon custard, lemonade, pink lemonade, lemon-lime, lime, malt, malted milk, mango, mango-pineapple, maple, margarita, marshmallow, meat type, condensed milk, cooked milk, mint, mirepoix, mocha, mochacinna, molasses, mushroom, sauteed mushroom, muskmelon, nectarine, neapolitan, green onion, sauteed onion, orange, orange cordial, orange creamsicle, orange creme, orange peach mango, orange strawberry banana, creamy orange, mandarin orange, orange-passion-guava, orange-pineapple, papaya, passion fruit, peach, peach mango, peanut, roasted peanut, pear, pecan danish type, pecan praline, pepper, peppermint, pimento, pina colada, pina colada/pineapple-coconut, pineapple, pineapple-orange, pistachio, pizza, pomegranate, pork fat type, baked potato, prune, punch, citrus punch, tropical punch, cherry fruit punch, grape punch, raspberry, black raspberry, blue raspberry, red raspberry, raspberry-blackberry, raspberry-ginger ale, raspberry-lime, roast type, root beer, rum, sangria, sarsaparilla, sassafras, sausage, sausage pizza, savory, seafood, shrimp, hickory smoke, mesquite smoke, sour, sour cream, sour cream and onion, spearmint, spicy, strawberry, strawberry margarita, jam type strawberry, strawberry-kiwi, burnt sugar, sweet & sour, tallow, tamarind, tangerine-lime, tangerine, tea, tequila type, toffee, triple sec, tropical fruit mix, turkey, tutti frutti, vanilla, vanilla cream, vanilla custard, french vanilla, vegetable, vermouth, vinegar, balsamic vinegar, watermelon, whiskey, wildberry, wine, and yogurt, and the like. Other examples of flavors are found in 21 C.F.R. §§ 172.510, 172.515, 172.520, 172.530, 172.535, 172.575, 172.580, and 172.585, which are hereby fully incorporated by reference. A variety of food grade flavors are commercially-available from Sensient Flavors Inc. in Indianapolis, Ind., Givaudan SA in Cincinnati, Ohio, and International Flavors & Fragrance in New York, N.Y.

Examples of taste maskants may include, but are not limited to, Smoothenols™ (available from Sensient Flavors, Inc.).

In some embodiments, the edible coating has a solids content that enhances the uniformity of the coating. In some embodiments, for example, preparing the solids at about 6% to about 8% lowers the viscosity, and when applying the film onto the edible substrate, the uniformity of the coating is increased. Thus, the film may be created with better rheological properties and uniform distribution of colors, including color tone.

In some embodiments, the edible coating comprises a solids content that enhances the uniformity of the coating. In some embodiments, the edible coating composition may have a solids content of at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, the edible coating composition may have a solids content of at most about 100%, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 9%, at most about 8%, at most about 7%, at most about 6%, at most about 5%, at most about 4%, at most about 3%, at most about 2%, or at most about 1%. This includes embodiments in which the edible coating composition may have solids contents ranging from about 0.1% to about 100%, such as solids contents ranging from about 0.5% to about 20.0%, or solids contents ranging from about 1.0% to about 15.0%.

The edible coating composition may have a viscosity of at least about 50 cP, at least about 100 cP, at least about 150 cP, at least about 200 cP, at least about 225 cP, at least about 250 cP, or at least about 275 cP. The edible coating composition may have a viscosity of at most at most about 400 cP, at most about 350 cP, at most about 335 cP, at most about 325 cP, at most about 300 cP, at most about 200 cP, or at most about 100 cP. This includes embodiments in which the edible coating composition may have viscosities ranging from about 200 cP to about 400 cP, such as viscosities ranging from about 225 cP to about 375 cP, or viscosities ranging from about 250 cP to about 300 cP.

Viscosity of the composition may be assessed by any suitable method such as, for example, using a Brookfield viscometer equipped with a UL Adapter Assembly and UL Spindle (available from Brookfield Engineering Labs., Inc.). The edible coating composition or other test sample is poured into a clean graduated cylinder or other sample container, and the spindle is immersed in the sample. After starting the viscometer motor, the speed is adjusted to achieve a percent torque between 50-70%, and the viscosity (cP) of the sample is recorded.

### 2. Edible Coatings

Edible coatings may be formed by applying the edible coating compositions to a substrate. The edible coatings disclosed herein may comprise the components of the edible coating compositions minus any components that are removed in the process of preparing the edible film coating. The edible coatings disclosed herein may be formed on any suitable substrate.

In some embodiments, the edible coatings may comprise the components of the edible coating compositions minus any components that evaporate in the process of preparing the edible film coating. In some embodiments, the edible coatings may comprise the non-water components of the edible coating composition.

In some embodiments, the edible coatings may have reduced moisture uptake in a humid environment compared to conventional HPMC coatings. Moisture uptake is a measure of the amount of water taken up as measured by weight gain of a sample that has been placed in a controlled environment of a set humidity. Moisture uptake can be measured by any suitable method known to one of skill in the art. Examples of procedures for measuring moisture uptake include, but are not limited to, exposing a coated substrate to 60% relative humidity at 25° C. (“storage condition H2”) or 75% relative humidity at 40° C. (“storage condition H4”), or 40-60% relative humidity at 20° C. (room conditions) and comparing the initial mass or weight of the sample to the mass or weight at various time intervals, such as 5 minutes, 30 minutes, 1 hour, 2 hours, 6 hours, 12 hours, 18 hours, 24 hours, 36 hours, 48 hours, 72 hours, 5 days, 7 days, 10 days, 14 days, 21 days, 25 days, 1 month, 2 months, 3 months, 6 months, 1 year, and the like. Stable mass or weight indicates a low moisture uptake. Without wishing to be limited by any particular theory, it is believed that the physical dimensions of an edible coating increase at a rate that corresponds to the moisture uptake. Accordingly, the moisture uptake can be investigated by measuring the physical size of the edible coating at various time intervals, as indicated above.

In some embodiments, a substrate may be coated with an edible coating composition to form an edible coating at a 1% weight gain, a 2% weight gain, a 3% weight gain, a 4% weight gain, a 5% weight gain, a 6% weight gain, a 7% weight gain, a 8% weight gain, a 9% weight gain, or a 10% weight gain compared to the uncoated substrate.

When exposed to storage condition H2, H4, or room conditions for a period of time equaling or exceeding 5 minutes, 30 minutes, 60 minutes, 90 minutes, 1 hour, 2 hours, 6 hours, 12 hours, 18 hours, 24 hours, 36 hours, 48 hours, 72 hours, 5 days, 7 days, 10 days, 14 days, 21 days, 25 days, 1 month, 2 months, 3 months, 6 months, 1 year, and the like, as measured by a VTI-SA Sorption Analyzer (available commercially from TA Instruments, New Castle, DE), the coated substrate or edible coating may exhibit a moisture uptake of at most about 4.0%, at most about 3.0%, at most about 2.5%, at most about 2.0%, at most about 1.9%, at most about 1.8%, at most about 1.7%, at most about 1.6%, at most about 1.5%, at most about 1.4%, at most about 1.3%, at most about 1.2%, at most about 1.1%, at most about 1.0%, at most about 0.95%, at most about 0.90%, at most about 0.85%, at most about 0.80%, at most about 0.75%, at most about 0.70%, at most about 0.65%, at most about 0.60%, at most about 0.55%, at most about 0.50%, at most about 0.45%, at most about 0.40%, at most about 0.35%, at most about 0.30%, at most about 0.25%, at most about 0.20%, at most about 0.15%, at most about 0.10% or at most 0.001%.

In some embodiments, the coated substrate or edible coating may have a moisture uptake, as measured by weight gain over time after exposure to storage condition H2, H4, or room conditions for the above-mentioned time durations, of at most about 0.1 grams per hour (g/hr), at most about 0.05 g/hr, at most about 0.04 g/hr, at most about 0.03 g/hr, at most about 0.02 g/hr, at most about 0.015 g/hr, at most about 0.01 g/hr, at most about 0.009 g/hr, at most about 0.008 g/hr, at most about 0.007 g/hr, at most about 0.006 g/hr, at most about 0.005 g/hr, at most about 0.004 g/hr, at most about 0.003 g/hr, at most about 0.002 g/hr, at most about 0.001 g/hr, or at most about 0.0005 g/hr. In some embodiments, the coated substrate or edible coating may have a moisture uptake, as measured by weight gain over time after exposure to storage condition H2, H4, or room conditions for the above-mentioned time durations, of at most about 0.01 inches per hour (in/hr), at most about 0.005 in/hr, at most about 0.004 in/hr, at most about 0.003 in/hr, at most about 0.002 in/hr, at most about 0.0015 in/hr, at most about 0.001 in/hr, at most about 0.0009 in/hr, at most about 0.0008 in/hr, at most about 0.0007 in/hr, at most about 0.0006 in/hr, at most about 0.0005 in/hr, at most about 0.0004 in/hr, at most about 0.0003 in/hr, at most about 0.0002 in/hr, at most about 0.0001 in/hr, or at most about 0.00005 in/hr.

In some embodiments, the edible coatings may have improved slip properties. Slip is a measure of the friction between surfaces, generally surfaces of coated dosage units. Improved slip indicates that less force is required. Additionally, improved slip properties may improve the swallowability and subject compliance of the edible coatings. The coatings can be tested based on the following: overall appearance, color/glossiness, texture/feel, mouth feel—taste perception, mouth feel—swallowability, slip, and mouth feel—swallowability, tablet shape, and feel. Additionally, swallowability may be examined by radioactivity (e.g., scintigraphy), as well as “synthetic esophagus” methods.

Slip can be measured by any suitable method known to one of skill in the art. Examples of procedures for measuring slip include, but are not limited to, smoothness measurements, among others.

Smoothness of the edible coatings may be measured by utilizing a TA.XT.Plus Texture Analyzer with a vertical friction rig serving as a force arm. The probe height and force may be calibrated and the return force arm positioned 1 mm from the bottom of the container. A non-friction gliding object, such as a piece of paper tissue, may be attached to the vertical probe of the rig using heavy-duty double-sided tape. A tablet or other substrate coated with an edible coating may be attached to the vertical wall of the rig using heavy-duty double-sided tape. A weight (e.g., 100 g) is added to the horizontal plate of the probe, and incremental increases of force are applied to the non-friction glidant in contact with the edible coating until the glidant slips or moves to measure the tension force.

A smooth edible coating may exhibit a slip at a tension of at least about 10 g, at least about 20 g, or at least about 30 g tension force. A smooth edible coating may exhibit a slip at a tension of less than about 100 g, less than about 90 g, less than about 80 g, less than about 70 g, less than about 60 g, less than about 50 g, less than about 40 g, less than about 30 g, or less than about 20 g friction force. A smooth edible coating may exhibit a slip at a friction force from about 10 g to about 100 g, a friction force from about 20 g to about 70 g, a friction force from about 30 g to about 50 g, a friction force from about 35 g to about 45 g, or a friction force from about 35 g to about 40 g.

In some embodiments, the coatings may exhibit high hardness. Hardness can be measured by any suitable method known to one of skill in the art. Examples of procedures for measuring hardness include, but are not limited to, Dr. Schleuniger Pharmatron 6D tablet tester using testing method set at kilopond (kp) force. Coatings disclosed herein may be capable of increasing the hardness of softer substrates upon coating or maintaining the hardness of harder substrates upon coating. In some embodiments, the coatings may have a hardness of at least about 10 kp, at least about 11 kp, at least about 12 kp, at least about 13 kp, at least about 14 kp, at least about 15 kp, at least about 16 kp, at least about 17 kp, at least about 18 kp, at least about 19 kp, at least about 20 kp, at least about 21 kp, at least about 22 kp, at least about 23 kp, at least about 24 kp, at least about 25 kp, at least about 26 kp, at least about 27 kp, at least about 28 kp, at least about 29 kp, at least about 30 kp, at least about 31 kp, at least about 32 kp, at least about 33 kp, at least about 34 kp, at least about 35 kp, at least about 36 kp, at least about 37 kp, at least about 38 kp, at least about 39 kp, or at least about 40 kp. This includes a hardness of about 10 kp to about 40 kp, a hardness of about 15 kp to about 35 kp, or a hardness of about 20 kp to about 30 kp.

In some embodiments, the coatings may exhibit an immediate release profile. Examples of procedures for determining the release profile include, but are not limited to, immediate release disintegration tests, such as USP32/NF27 S2, Chapter 2040 (Disintegration and Dissolution of Dietary Supplements), Chapter 701 (Disintegration) testing criteria, and the like. In some embodiments, the coatings may provide for release (e.g., of contents contained therein) within a particular time period. The time period may be no longer than 120 minutes, 90 minutes, 60 minutes, 45 minutes, 30 minutes, 25 minutes, 20 minutes, 15 minutes, 10 minutes, 5 minutes, 3 minutes, or 2 minutes.

In some embodiments, the coatings may exhibit good adhesion to a substrate. Adhesion is a measure of how strongly the coating binds to the substrate. Adhesion of the coatings may be measured using a TA.XT.Plus Texture Analyzer (available from Texture Technologies Corp.) equipped with a 25 mm stainless steel cylindrical probe. A coated substrate is attached to the top of the flat platform of the texture analyzer using heavy-duty double-sided tape (available from 3M). Another piece of heavy-duty double-sided tape is pressed to the bottom of the cylindrical probe, and the probe is then compressed to 800 g force onto the coated substrate for 10 seconds. The probe is then pulled away from the coated substrate at a rate of 1 mm/second, measuring the tension force until either (1) the coating separates from the substrate, or (2) the tape separates from the coating. Where the coating remains on the substrate, the measured force equals the force required to pull the double-sided tape away from the coating, and the adhesion force of the coating to the substrate is thus greater than the measured adhesion force between the tape and the coating. An adherent coating may exhibit an adhesion force between the coating and the substrate of at least about 50 g force, at least about 100 g force, at least about 200 g force, at least about 400 g force, at least about 600 g force, at least about 700 g force, at least about 800 g force, at least about 900 g force, or at least about 1000 g force.

In some embodiments, coatings may exhibit low friability. Friability can be measured by any suitable method known to one of skill in the art. Examples of procedures for measuring friability include, but are not limited to, Dr. Schleuniger Pharmatron method FTV-2, U.S. Pharmacopeia Chapter 1216 Tablet Friability, measuring the weight difference after exposing tablets to rotating drums at 25 RPM+1 RPM for 100 revolutions, and the like. In some embodiments, the friability may be at most about 1%, at most about 0.1%, or at most about 0.01%.

In some embodiments, the coatings may exhibit good clarity. Examples of procedures for measuring clarity include, but are not limited to, measuring the lightness, whiteness, or yellowness of an uncoated sample and comparing to the respective lightness, whiteness, or yellowness of a coated sample. Smaller changes correspond to higher clarity. Lightness can be assessed by any suitable method such as, for example, measuring the Lightness Index according to ASTM Method E313 with a D65/10° illumination source (referred to as “E313 [D65/10]”) using a LabScan XE spectrophotometer (available from HunterLab, Inc.). A sample is loaded into the instrument's sample port and scanned, and the Lightness Index (E313 [D65/10]) value is calculated using measurements taken on the CIE L*a*b* color scale. The resulting Lightness Index value is used to assess lightness of the sample. Whiteness can be assessed by any suitable method, such as, for example, measuring the Whiteness Index according to ASTM Method E313 with a D65/10° illumination source (referred to as “WI E313 [D65/10]”) using a LabScan XE spectrophotometer (available from HunterLab, Inc.). A sample substrate coated with a film coating is loaded into the instrument's sample port and scanned, and the Whiteness Index (WI E313 [D65/10]) value is calculated using measurements taken on the CIE L*a*b* color scale. The resulting Whiteness Index value is used to assess whiteness of the sample. Yellowness can be assessed by any suitable method, such as, for example, measuring the Yellowness Index according to ASTM Method E313 with a D65/10° illumination source (referred to as “YI E313 [D65/10]”) using a LabScan XE spectrophotometer. A sample substrate coated with a film coating is loaded into the instrument's sample port and scanned, and the resulting YI E313 [D65/10] value is used to assess yellowness of the sample. In some embodiments, the change in lightness, whiteness, or yellowness may be less than about 50%, less than about 25%, less than about 20%, less than about 15%, less than about 10%, less than about 5%, or less than about 1%.

In some embodiments, the coatings may have high whiteness and/or high opacity. Whiteness can be assessed as described above. Opacity can be assessed by evaluating the Whiteness Index achieved upon coating one or more colored substrate cores with edible coatings as described herein. For example, when coating a colored substrate, the Whiteness Index will increase with additional weight gain until high and/or full opacity is reached. Upon reaching high and/or full opacity, the Whiteness Index may level off relative to further increases in weight gain. Suitably, the disclosed film coatings may provide high and/or full opacity at a weight gain of less than about 10%, less than about 9%, less than about 8%, less than about 7%, less than about 6%, less than about 5%, less than about 4%, less than about 3%, or less than about 2% weight gain. For example, the disclosed edible coatings may provide high and/or full opacity at weight gains ranging from about 1% to about 10%, such as weight gains ranging from about 3% to about 9%, or weight gains ranging from about 2% to about 6%. In some embodiments, the disclosed film coating compositions, film coating suspensions, and/or film coatings comprise an opacifying agent in amounts sufficient to provide a film coating with high whiteness and/or full/high opacity at low weight gain.

In some embodiments, applying the composition to an edible substrate produces a coated edible substrate, the edible substrate and the coated edible substrate each having a gloss, wherein the gloss of the coated edible substrate may be from about 0% to about 500% greater than the gloss of the edible substrate. In some embodiments, the gloss of the coated edible substrate may be at least about 5% greater, at least about 10% greater, at least about 50% greater, at least about 100% greater, at least about 150% greater, at least about 200% greater, at least about 300% greater, at least about 400, or at least about 500% greater.

In some embodiments, applying the composition to an edible substrate produces a coated edible substrate, the edible substrate and the coated edible substrate each having a reflectance spectrum having a peak wavelength, a peak width, and an integrated area, wherein the peak wavelength, peak width, or integrated area may vary by less than about 50% from edible substrate to coated edible substrate. In some embodiments, the peak wavelength, peak width, or integrated area may vary by less than about 50%, less than about 40%, less than about 30%, less than about 25%, less than about 20%, less than about 15%, less than about 10%, less than about 5%, less than about 4%, less than about 3%, less than about 2%, less than about 1%, less than about 0.5%, or less than about 0.1%.

In some embodiments, applying the composition to an edible substrate produces an edible coating on the edible substrate, the edible coating having a transmission haze of less than about 50% as measured by ASTM D1003-95, “Standard Test Method for Haze and Luminous Transmittance of Transparent Plastics” or a reflection haze of less than about 50% as measured by ASTM E430-91, “Standard Test Methods for Measurement of Gloss of High-Gloss Surfaces by Goniophotometry.” Gloss may be measured using Novocure equipment (Rhopoint Instruments, UK).

In some embodiments, the coatings may exhibit good stability. Stability indicates the ability for a coating to remain functional under normal or abnormal storage conditions. One example of a means of testing stability is exposing a coated substrate to 60% relative humidity at 25° C. (“storage condition H2”), 75% relative humidity at 40° C. (“storage condition H4”), or room conditions and evaluating a measurable property. Changes in the measurable property over time are an indication of lack of stability with respect to that measurable property and the particular conditions studied. In some embodiments, the measurable property may change by less than about 50%, less than about 25%, less than about 20%, less than about 15%, less than about 10%, less than about 5%, less than about 1%, less than about 0.1%, or less than about 0.01% over a period of time of at least about 30 minutes, at least about 1 hours, at least about 6 hours, at least about 12 hours, at least about 24 hours, at least about 7 days, at least about two weeks, at least about one month, at least about three months, at least about six months, or at least about one year. Examples of measurable properties include, but are not limited to, whiteness, brightness, yellowness, clarity, opacity, gloss, size, smoothness, friability, transmission haze, adhesion, hardness, and moisture barrier.

### 3. Methods of Making Edible Coating Compositions

The methods of making the edible coating compositions may comprise mixing cellulose film former, a plasticizer, and a lubricant to form a mixture, and optionally combining the mixture with water. Other ingredients may be optionally added to provide extended attributes, such as colors, flavors, and taste maskants, for example.

In general, the mixing and combining steps may be performed by any suitable method using any suitable apparatus known to one of skill in the art. Examples of suitable apparatuses for combining the ingredients of the compositions disclosed herein include, but are not limited to, plow mixers, low-shear overhead mixers in solution preparation, multi-action blades on overhead stirrers, and the like. Suitable mixers are available commercially from Littleford Day, Inc. (Florence, KY) and Lodige Mixers (Paderborn Germany), among others.

In one aspect, the method comprises: (1) weighing the desired amount of water into an appropriately sized container; (2) stirring the water with a high vortex; (3) mixing the dry ingredients and adding the dry ingredients into the vortex wall with rapid stirring; (4) stirring the resulting mixture at the rapid stirring rate for 5 minutes; and (5) stirring the resulting mixture at a stirring rate that is 25% less than the rapid stirring rate for 25 minutes.

In another aspect, the method comprises adding glycerin to stirring water, then adding IPMC to the stirring water. In yet another aspect, the method comprises adding glycerin and oil to stirring water, then adding IPMC to the stirring water.

The compositions described herein can be manufactured using techniques and equipment that are known and commonly used in the art. Manufacturing steps such as order of component addition, mixing temperatures (heating and/or cooling), mixing time, mixing speed, etc. can be driven either by formulation or equipment requirements, or both. A number of parameters can be modified during the manufacturing process without substantial effect on the efficacy of the resulting product. The manufacturing methods and processes can further include separate steps for validating the resulting composition (e.g., the total amounts, ratios, and even distribution of components in the composition, etc.).

In embodiments, the compositions described herein are sprayed onto a substrate under the conditions in Table 1. The parenthetical numbers in Table 1 indicate some specific values which were successfully tested. The ability to obtain the targeted air flow with the disclosed compositions is particularly desirable, which at least in part allows for a good quality coating.

TABLE 1
Example of spraying parameters with a 0.5 L vector pan, 1 gun, #10 tip
(w44019), #27 cap (w44183), and Schlick e45738 ring.

| | |
|---|---|
| Pan Speed Target (rpm) | 15-20 (18) |
| Inlet Target (° C.) | 48-56 (52) |
| Coater Airflow (cfm) | 50-58 (50) |
| Atom. Target (psi) | 19.5 |
| Exhaust Target (° C.) | 40-44 (40.5) |

The edible coating is easily prepared, which increases efficiency of coating, thus providing a more cost-effective coating. In some embodiments, the edible coating may be ready to use in less than about 90 minutes, less than about 85 minutes, less than about 80 minutes, less than about 75 minutes, less than about 70 minutes, less than about 65 minutes, less than about 60 minutes, less than about 55 minutes, less than about 50 minutes, less than about 45 minutes, less than about 40 minutes, less than about 35 minutes, or less than about 30 minutes.

### 4. Methods of Using Edible Coating Compositions

In one aspect, the edible coating composition may be applied directly or indirectly to an edible substrate to produce a coated edible substrate. The edible substrate may or may not be pre-coated with other coatings. In other words, in some embodiments no pre-coat or subcoat is needed—again, the edible coating composition may be applied directly to the edible substrate, food, pharmaceutical, or nutraceutical. In some embodiments, the edible substrate may be a nutraceutical or pharmaceutical dosage unit, such as a capsule, tablet, or softgel. In other embodiments, the edible substrate may be a nutritional supplement, a pharmaceutical, a tablet, a capsule, a softgel, a granule, a microparticle, a nanoparticle, a seed, a sugar sphere, gum chew base, chewable gel, or a gummy substrate. Other suitable edible substrates would be known to one of ordinary skill in the art.

The edible coating compositions and edible coatings may be used in food, pharmaceutical, or nutraceutical applications intended for use in mammals, including, without limitation, rodents, canines, felines, non-human primates, ungulates, and humans. The edible coating compositions and edible coatings may be used to coat pharmaceutical or non-pharmaceutical dosage units.

The disclosed edible coating compositions may, at a suitable concentration which is system dependent, be applied (e.g., sprayed) to form an edible coating on an edible substrate. In certain embodiments, the edible coating compositions may be applied using commercially-available equipment. Equipment known in the industry for standard tablet coating are Thomas Engineering, Inc., Vector Corporation Hi-Coater®, O'Hara Technologies, and Coating Systems International TechniCota tablet coating systems, to name a few.

In some embodiments, the methods may include applying an edible coating composition to an edible substrate to a weight gain of at least about 1%, at least about 2%, at least about 3%, at least about 4%, at least about 5%, at least about 6%, at least about 7%, at least about 8%, at least about 9%, or at least about 10%. In some embodiments, the methods may include applying an edible coating composition to a substrate to a weight gain of at most about 10%, at most about 9%, at most about 8%, at most about 7%, at most about 6%, at most about 5%, at most about 4%, at most about 3%, or at most about 2%. For example, the methods may include applying an edible coating composition to a substrate to a weight gain ranging from about 2% to about 6%.

In some embodiments, the film coating suspension may be applied to a substrate by loading the substrate into a vented coating pan, such as, for example, a 15-inch, 24-inch, 48-inch, or 60-inch side-vented coating pan.

The inlet temperature may be at least about 45° C., at least about 50° C., at least about 55° C., at least about 58° C., at least about 60° C., at least about 65° C., or at least about 70° C. The inlet temperature may be less than about 80° C., less than about 75° C., less than about 70° C., less than about 65° C., or less than about 60° C. This includes, for example, about 50° C. to about 80° C., about 50° C. to about 65° C., or about 55° C. to about 75° C. The outlet temperature may be at least about 35° C., at least about 40° C., at least about 44° C., or at least about 45° C. The outlet temperature may be less than about 65° C., less than about 60° C., less than about 50° C., or less than about 46° C. This includes, for example, about 44° C. to about 65° C., about 44° C. to about 46° C., or about 45° C. to about 60° C.

The atomizing air pressure may be at least about 20 psi, at least about 21 psi, at least about 25 psi, at least about 30 psi, at least about 35 psi, at least about 40 psi, at least about 45 psi, at least about 50 psi, or at least about 55 psi. The atomizing air pressure may be less than about 75 psi, less than about 70 psi, less than about 65 psi, less than about 60 psi, less than about 55 psi, less than about 50 psi, less than about 40 psi, or less than about 30 psi. This includes, for example, about 20 to about 75 psi, about 23 to about 25 psi, about 25 to about 30 psi, about 35 to about 55 psi, or about 50 to about 70 psi.

In some embodiments, the spray rate may be at least about 10 g/min, at least about 15 g/min, at least about 20 g/min, at least about 25 g/min, at least about 30 g/min, at least about 35 g/min, at least about 40 g/min, at least about 50 g/min, at least about 75 g/min, at least about 100 g/min, at least about 125 g/min, at least about 150 g/min, at least about 175 g/min, at least about 200 g/min, at least about 225 g/min, at least about 250 g/min, at least about 275 g/min, at least about 300 g/min, at least about 325 g/min, at least about 350 g/min, at least about 375 g/min, at least about 400 g/min, at least about 425 g/min, at least about 450 g/min, at least about 475 g/min, at least about 500 g/min, at least about 525 g/min, at least about 550 g/min, or at least about 575 g/min. In some embodiments, the spray rate may be at most about 600 g/min, at most about 575 g/min, at most about 550 g/min, at most about 525 g/min, at most about 500 g/min, at most about 475 g/min, at most about 450 g/min, at most about 425 g/min, at most about 400 g/min, at most about 375 g/min, at most about 350 g/min, at most about 325 g/min, at most about 300 g/min, at most about 275 g/min, at most about 250 g/min, at most about 225 g/min, at most about 200 g/min, at most about 175 g/min, at most about 150 g/min, at most about 125 g/min, at most about 100 g/min, at most about 95 g/min, at most about 90 g/min, at most about 85 g/min, at most about 80 g/min, at most about 75 g/min, at most about 70 g/min, at most about 65 g/min, at most about 60 g/min, at most about 55 g/min, at most about 50 g/min, at most about 45 g/min, at most about 40 g/min, at most about 35 g/min, at most about 30 g/min, at most about 25 g/min, at most about 20 g/min, at most about 19 g/min, at most about 18 g/min, at most about 17 g/min, at most about 16 g/min, at most about 15 g/min, at most about 14 g/min, at most about 13 g/min, at most about 12 g/min, at most about 11 g/min, or at most about 10 g/min. This includes, for example, about 10 g/min to about 20 g/min, about 10 g/min to about 12 g/min, about 12 g/min to about 15 g/min, about 15 g/min to about 20 g/min, about 35 g/min to about 75 g/min, about 35 g/min to about 45 g/min, about 45 g/min to about 55 g/min, about 55 g/min to about 75 g/min, about 175 g/min to about 350 g/min, about 175 g/min to about 275 g/min, about 275 g/min to about 325 g/min, about 325 g/min to about 350 g/min, about 275 g/min to about 600 g/min, about 275 g/min to about 450 g/min, about 450 g/min to about 550 g/min, or about 550 g/min to about 600 g/min.

A higher spray rate may require the performance of a curing step after spray application. Without wishing to be limited by any particular theory, it is believed that a higher spray rate results in more water within the resulting coating. Therefore, without curing, the excess water within a coating produced by a higher spray rate may penetrate the core. In some embodiments, the methods may include curing the edible coating for at least about 1 minute, at least about 2 minutes, at least about 3 minutes, at least about 4 minutes, at least about 5 minutes, at least about 6 minutes, at least about 7 minutes, at least about 8 minutes, at least about 9 minutes, or at least about 10 minutes. In some embodiments, the methods may include curing the edible coating for at most about 30 minutes, at most about 25 minutes, at most about 20 minutes, at most about 15 minutes, at most about 14 minutes, at most about 13 minutes, at most about 12 minutes, at most about 11 minutes, at most about 10 minutes, at most about 9 minutes, at most about 8 minutes, at most about 7 minutes, at most about 6 minutes, at most about 5 minutes, at most about 4 minutes, at most about 3 minutes, or at most about 2 minutes. This includes embodiments in which the methods include curing the edible coating for times ranging from about 1 minute to about 30 minutes, such as times ranging from about 2 minutes to about 20 minutes, or times ranging from about 5 minutes to about 15 minutes.

The coating time may be at least about 5 minutes, at least about 30 minutes, at least about 1 hour, at least about 2 hours, or at least about 3 hours. The coating time may be at most about 3 hours, at most about 2 hours, at most about 1 hour, or at most about 30 minutes. The coating time may range from about 5 minutes to about 3 hours, such as from about 10 minutes to about 3 hours.

The film coating composition may be allowed to dry, forming a film coating on the substrate. Exemplary coating parameters are provided in Table 2.

TABLE 2

| | Pan Size | | | |
|---|---|---|---|---|
| | 15 inch | 24 inch | 48 inch | 60 inch |
| Air volume (cfm) | 225-250 | 250-320 | 1800-2400 | 3800-4200 |
| Inlet temp. (° C.) | 50-65 | 50-65 | 55-75 | 55-80 |
| Outlet temp. (° C.) | 44-46 | 44-46 | 45-60 | 46-65 |
| Curing temp. (° C.)* | 42-44 | 42-44 | 42-50 | 42-50 |
| Pre-warm temp. (° C.) | 38-40 | 38-40 | 40-45 | 40-45 |
| Spray rate (g/min) | 10-20 | 35-75 | 175-350 | 275-600 |
| | 10-12** | 35-45** | 175-275** | 275-450** |
| | 12-15*** | 45-55*** | 275-325*** | 450-550*** |
| | 15-20**** | 55-75**** | 325-350**** | 550-600**** |
| Atomizing air pressure (psi) | 23-25 | 25-30 | 35-55 | 38-55 |
| Pattern air pressure (psi) | 20 | 20 | 35-50 | 35-50 |
| No. of guns | 1 | 1 | 3-4 | 4-6 |
| Pan speed (rpm) | 10-15 | 10-15 | 4-10 | 4-8 |
| Pan charge weight (kg) | 1-3 | 12-14 | 120-200 | 250-350 |

*Curing temperature if curing necessary or desired.
**Range of spray rate in which curing is likely unnecessary to achieve moisture barrier properties.
***Range of spray rate in which curing may be necessary to achieve moisture barrier properties.
****Range of spray rate in which curing is likely necessary to achieve moisture barrier properties.

In some embodiments, a coating pan may be charged with a substrate such as, for example, capsules, tablets, and/or softgels. In some embodiments, the substrate may be warmed to about 33° C. to about 50° C. Further processing parameters may be as set forth herein.

### EXAMPLES

The following examples are presented to illustrate the present disclosure and to assist one of ordinary skill in making and using the same. The examples are not intended in any way to otherwise limit the scope of the disclosure.

Materials. As used herein, the following terms have the corresponding meanings. “HPMC 5 SE” denotes Spectracel™ 5-SE (available commercially from Sensient Technologies, Inc., St. Louis, MO). “HPMC 3 SE” denotes Spectracel™ 3-SE (available commercially from Sensient Technologies, Inc., St. Louis, MO). “HPMC 15 SE” denotes Spectracel™ 15-SE (available commercially from Sensient Technologies, Inc., St. Louis, MO). “HPMC” may include Mantrocel™ E-5 HPMC and Mantrocel™ E-6 HPMC (available commercially from Mantrose-Haeuser Co., Inc., Westport, CT). Sunflower oil may be obtained from Stratas Foods under the trade name Trisun 80 High Oleic Sunflower Oil RBWD.

Unless otherwise indicated, example compositions were prepared by mixing the ingredients in a Littleford Plow Mixer. Solutions were prepared by using low-shear overhead mixers according to the methods described herein.

Testing was performed on coating samples including various levels of oil (e.g., sunflower oil) into HPMC/glycerin formulations. Surprisingly, adding glycerin and some oil significantly affected the water vapor permeability (WVP) to afford even better barrier property outcomes.

Four of the tested formulations are shown in Table 3. Formula 0 is a control sample with no oil. A broad range of HPMC grades were tested for each formulation in Table 3, such as HPMC 3cp, HPMC 5cp, HPMC 6cp, and HPMC 15cp, for example. HPMC 15cp, HPMC 6cp, and HPMC 5cp are preferable.

TABLE 3
Formulations with 10% solutions solids

| | | | Sunflower |
|---|---|---|---|
| Formula | HPMC | Glycerin | Oil |
| 0 | 9.2 g | 0.8 g | 0.0 g |
| 1 | 9.2 g | 0.7 g | 0.1 g |
| 2 | 9.2 g | 0.6 g | 0.2 g |
| 3 | 9.2 g | 0.5 g | 0.3 g |
| 4 | 9.2 g | 0.4 g | 0.4 g |

The percent moisture uptake data was evaluated on a VTI Sorption Analyzer. The instrumental criteria used was that of 75% moisture uptake on a gradient at 40° C. (within 0.01° C.) for one cycle. Certain other samples tested at 1% weight gain are as follows: using a coating with a mixture of long chain and medium chain HPMC, use testing at 8% solution solids resulted in a measured moisture uptake of 0.074%/min; and using a coating with long-chain HPMC, use testing at 8% solution solids resulted in a moisture uptake of 0.084%/min. The same procedure will be used to evaluate colored tablets for moisture uptake. The colored tablets have the same formula as those tested at 1% weight gain, described above, but with the addition of riboflavin.

Additionally, tablets were coated using placebos, arginine, and vitamin cores using 8% to 10% solution solids. The conditions used to coat the tablets were with an exhaust at 40 to 42 dC, and a slow to medium spray rate in order to sufficiently remove any moisture. The air volume was also maintained to sufficiently remove any excess moisture with preferred conditioned air volume.

Work was also done to test the slip effect of various percent sunflower oil additions into HPMC/glycerin films, although a visual analysis was sufficient to determine the absence of certain coating defects. For example, visual analysis showed that there was good edge coverage, no panscruffing, and no logo fill. It should be noted that with each increase in oil, a decrease in glycerin was used to maintain the same polymer morphology for the compositions used for slip testing (as exemplified in Table 3).

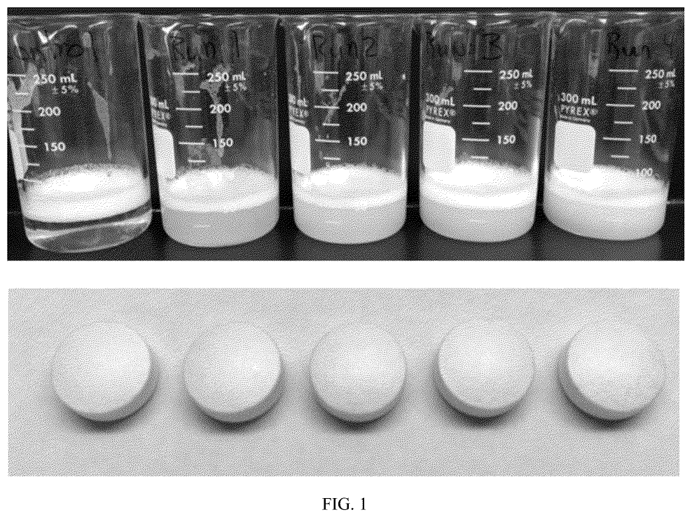

Blends were prepared in a non-GMP R&D Applications laboratory. The tablets were coated with the blends under the same conditions—non-GMP. Solutions of 8% to 10% revealed an increasing cloudiness with increasing amounts of sunflower oil (FIG. 1, top).

A HPMC/glycerin formulation from long chain HPMC was used in evaluating the effectiveness of slip film attributes when adding 1%, 2%, 3%, and 4% sunflower oil and replacing the glycerin, respectively, to ensure that the same polymer quantity was present in the formulation. The coating was applied to Miralac Sugar Free Antacid tablets containing 420 mg of calcium carbonate (FIG. 1, bottom).

The stability testing was done on the samples with the coated tablets placed into vials, the vials being closed with a screw top lid and kept in three different conditions: H4 at 40° C./75% RH, H2 at 25° C./60%, and RT at 20° C./45%. After 1 week, none of the coated tablets showed any signs of degradation.

The coating compositions which included 1% to 4% oil showed an improvement in spray delivery when atomizing onto the tablet surface. Spray efficiencies increased with decreased spray times from 13.6 minutes to 12.8 minutes, which equates to saving at least 6% time in processing! In other words, it was surprisingly discovered that the disclosed coating compositions had markedly improved slip and film deposition properties, as well as an increased film drying time, due at least in part to better particle coalescence.

Disintegration testing (DT) indicated a decrease in performance with 4% oil inclusion, and therefore, less than or equal to 3% oil inclusion is generally preferred, more preferably 2% to 3% oil inclusion. However, samples with 4% oil inclusion showed a passable performance (Table 4).

TABLE 4
Time in seconds for the film to start to peel or bubble
from the core. SGF stands for simulated gastric fluid.

| Coating level | In DI water | In SGF |
|---|---|---|
| of oil inclusion | 37° C. bath | 37° C. bath |
| Control (no oil) | 94 sec | 12 sec bubbles |
| 1% oil in coating | 78 sec | 12 sec bubbles |
| 2% oil in coating | 57 sec | 19 sec bubbles |
| 3% oil in coating | 57 sec | 30 sec bubbles |
| 4% oil in coating | 138 sec | 30 sec bubbles |

Also performed were total color difference tests (DIELab calculations), which showed Delta E (dE) changes below 1 for all oil-inclusive samples, indicating no visual change could be detected by the human eye (Table 5). dE is a single number that expresses magnitude (i.e., size, degree, amount) of difference between two colors.

TABLE 5
dE imaging results after 1 week

| ID | L* | a* | b* | dL | da* | db* | dE* | STDEV |
|---|---|---|---|---|---|---|---|---|
| 129-061 | 95.46 | 0.35 | 2.91 | 95.46 | 0.35 | 2.91 | — | — |
| No coat RT | 96.81 | 0.52 | 3.57 | 1.36 | 0.18 | 0.66 | 1.52 | 0.125033 |
| No coat H2 | 96.46 | 0.55 | 3.86 | 1.00 | 0.20 | 0.95 | 1.40 | |
| No coat H4 | 96.22 | 0.61 | 4.35 | 0.76 | 0.26 | 1.44 | 1.65 | |
| HPMC/Gly RT | 95.69 | 0.31 | 2.44 | 0.23 | −0.03 | −0.47 | 0.53 | 0.700 |
| HPMC/Gly H2 | 95.77 | 0.37 | 2.26 | 0.31 | 0.03 | −0.65 | 0.72 | |
| HPMC/Gly H4 | 94.95 | 0.36 | 3.59 | −0.51 | 0.02 | 0.68 | 0.85 | |
| HPMC/Gly + 1% oil RT | 95.59 | 0.41 | 2.55 | 0.14 | 0.07 | −0.36 | 0.39 | 0.223681 |
| HPMC/Gly + 1% oil H2 | 95.89 | 0.37 | 2.21 | 0.43 | 0.02 | −0.71 | 0.83 | |
| HPMC/Gly + 1% oil H4 | 95.29 | 0.40 | 3.42 | −0.17 | 0.05 | 0.51 | 0.54 | |
| HPMC/Gly + 2% oil RT | 95.64 | 0.34 | 2.15 | 0.19 | 0.00 | −0.76 | 0.78 | 0.37899 |
| HPMC/Gly + 2% oil H2 | 95.87 | 0.41 | 2.32 | 0.41 | 0.06 | −0.60 | 0.73 | |
| HPMC/Gly + 2% oil H4 | 95.43 | 0.33 | 2.82 | −0.03 | −0.01 | −0.09 | 0.10 | |
| HPMC/Gly + 3% oil RT | 96.07 | 0.30 | 2.11 | 0.61 | −0.05 | −0.80 | 1.01 | 0.328075 |
| HPMC/Gly + 3% oil H2 | 96.02 | 0.38 | 2.16 | 0.56 | 0.04 | −0.75 | 0.94 | |
| HPMC/Gly + 3% oil H4 | 95.69 | 0.29 | 2.58 | 0.24 | −0.05 | −0.33 | 0.41 | |
| HPMC/Gly + 4% oil RT | 95.75 | 0.36 | 2.74 | 0.29 | 0.01 | −0.17 | 0.34 | 0.337095 |
| HPMC/Gly + 4% oil H2 | 96.00 | 0.35 | 2.31 | 0.55 | 0.01 | −0.61 | 0.82 | |
| HPMC/Gly + 4% oil H4 | 95.37 | 0.31 | 3.06 | −0.09 | −0.03 | 0.14 | 0.17 | |

Improved tablet slip during coating occurred with oil inclusion, particularly after 3% vegetable oil was incorporated into the film. Film slip was tested on a TX XT Texture Analyzer slip rig.

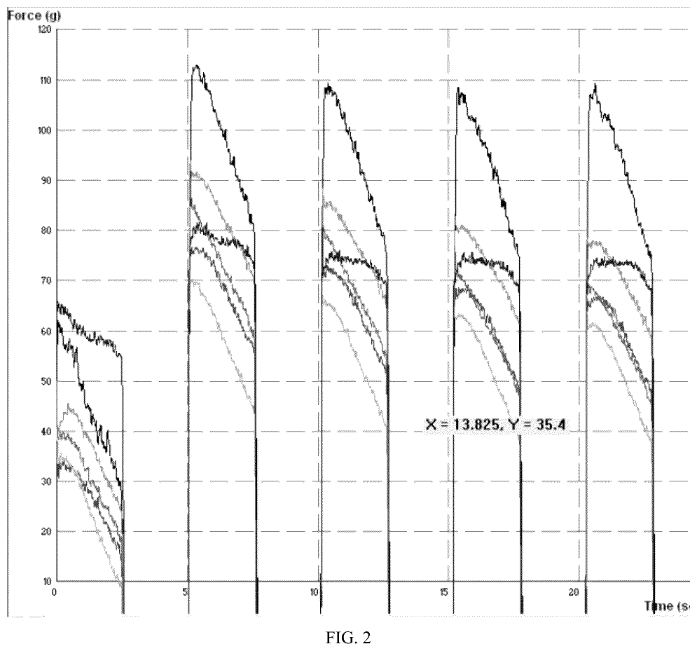

FIG. 2 shows results of a slip analysis. The top black line is a control sample without any coating, which shows the most significant drag. The blue line is a coating with 0% oil inclusion, which has the most jagged line without a downward slope on the downward trend. The red line is a sample with 1% oil inclusion. Similarly, the green, light blue, and purple lines are samples with 2%, 3%, and 4% oil inclusion, respectively.

For reasons of completeness, various aspects of the invention are set out in the following numbered clauses.

Clause 1. A composition for coating an edible substrate, the composition comprising:
about 85.0 wt % to about 95.0 wt % of a cellulose film former;
about 5.0 wt % to about 15.0 wt % of a plasticizer; and
about 1.0 wt % to about 4.0 wt % of a lubricant selected from sunflower oil, vegetable oil, hydrogenated vegetable oil, mineral oil, a fatty acid, or a combination thereof, relative to the total weight of non-water components of the composition.

Clause 2. A method of forming an edible coating on an edible substrate comprising:
spraying the composition of clause 1 onto the substrate.

Clause 3 The composition of clause 1 or the method of clause 2, wherein the cellulose film former comprises hydroxypropyl methyl cellulose (HPMC).

Clause 4. The composition or the method of any one of clauses 1-3, wherein the plasticizer comprises glycerin.

Clause 5. The composition or the method of any one of clauses 1-4, wherein the lubricant comprises sunflower oil.

Clause 6. The composition or the method of any one of clauses 1-5, wherein the composition comprises only one type of the plasticizer and/or only one type of lubricant.

Clause 7. The composition or the method of any one of clauses 1-6, wherein the cellulose film former is present in an amount of about 87.0 wt % to about 93.0 wt % relative to the total weight of non-water components of the composition.

Clause 8. The composition or the method of any one of clauses 1-7, wherein the plasticizer is present in an amount of about 6.0 wt % to about 10.0 wt % relative to the total weight of non-water components of the composition.

Clause 9. The composition or the method of any one of clauses 1-8, wherein the lubricant is present in an amount of about 2.0 wt % to about 3.0% relative to the total weight of non-water components of the composition.

Clause 10. The composition or the method of any one of clauses 1-9, wherein the ratio of the plasticizer to the lubricant by weight is less than or equal to 8.

Clause 11. The composition or the method of any one of clauses 1-10, wherein the composition is applied to an edible substrate to form an edible coating and the edible coating has a moisture uptake of less than or equal to 3% by weight relative to the total weight of the initial edible coating when exposed to 40° C. and 75% relative humidity for 60 minutes, as measured by a VTI-SA Sorption Analyzer.

Clause 12. The composition or the method of any one of clauses 1-11, wherein the composition is applied to an edible substrate to form an edible coating and the edible coating has a surface friction of less than or equal to 70 grams of force, as measured by a TX XT Texture Analyzer slip rig.

Clause 13. The composition or the method of any one of clauses 1-12, wherein the composition is applied to an edible substrate to form an edible coating and the edible coating starts to peel or bubble from the substrate in less than or equal to 80 seconds in a bath of deionized water at 37° C.

Clause 14. The composition or the method of any one of clauses 1-13, wherein the composition is applied to an edible substrate to form an edible coating and the edible coating starts to peel or bubble from the substrate in less than or equal to 60 seconds in a bath of deionized water at 37° C.

Clause 15. The composition or the method of any one of clause 1-14, wherein the composition is applied to an edible substrate to a weight gain of about 1% to about 3% of the edible substrate.

Clause 16. The composition or the method of any one of clauses 1-15, wherein the composition is sprayed onto a substrate and the spray time is less than 13.6 minutes under the spray conditions defined by Table 1.

Clause 17. The composition or the method of any one of clauses 1-16, wherein the composition is sprayed onto a substrate and the spray time is less than 12.8 minutes under the spray conditions defined by Table 1.

Clause 18. The composition or the method of any one of clauses 1-17, wherein the composition is sprayed onto a substrate and the spray time is at least 6% more efficient compared to an otherwise identical edible coating composition not comprising the lubricant.

Clause 19. The composition or the method of any one of clauses 1-18, wherein the edible substrate is a nutritional supplement, a pharmaceutical, a tablet, a capsule, a softgel, a granule, a microparticle, a nanoparticle, a seed, a sugar sphere, gum chew base, chewable gel, or a gummy substrate.

Clause 20. The composition or the method of any one of clause 1-19, wherein the lubricant is a non-allergen oil and/or a non-genetically modified organism (non-GMO) oil.

Clause 21. A nutritional supplement, a pharmaceutical, a tablet, a capsule, a granule, a microparticle, a nanoparticle, or a seed comprising an edible substrate and the composition of any of clauses 1 and 3-20 thereon.

1. A composition for coating an edible substrate, the composition comprising:
about 85.0 wt % to about 95.0 wt % of a cellulose film former about 5.0 wt % to about 15.0 wt % of a plasticizer; and
about 1.0 wt % to about 4.0 wt % of a lubricant selected from sunflower oil, vegetable oil, hydrogenated vegetable oil, mineral oil, a fatty acid, or a combination thereof, relative to the total weight of non-water components of the composition.

2. A method of forming an edible coating on an edible substrate comprising:
spraying the composition of claim 1 onto the substrate.

3. The composition of claim 1, wherein the cellulose film former comprises hydroxypropyl methyl cellulose (HPMC).

4. The composition or the method of claim 1, wherein the plasticizer comprises glycerin.

5. The composition or the method of claim 1, wherein the lubricant comprises sunflower oil.

6. The composition or the method of claim 1, wherein the composition comprises only one type of the plasticizer and/or only one type of lubricant.

7. The composition or the method of claim 1, wherein the cellulose film former is present in an amount of about 87.0 wt % to about 93.0 wt % relative to the total weight of non-water components of the composition.

8. The composition or the method of claim 1, wherein the plasticizer is present in an amount of about 6.0 wt % to about 10.0 wt % relative to the total weight of non-water components of the composition.

9. The composition or the method of claim 1, wherein the lubricant is present in an amount of about 2.0 wt % to about 3.0% relative to the total weight of non-water components of the composition.

10. The composition or the method of claim 1, wherein the ratio of the plasticizer to the lubricant by weight is less than or equal to 8.

11. The composition or the method of claim 1, wherein the composition is applied to an edible substrate to form an edible coating and the edible coating has a moisture uptake of less than or equal to 3% by weight relative to the total weight of the initial edible coating when exposed to 40° C. and 75% relative humidity for 60 minutes, as measured by a VTI-SA Sorption Analyzer.

12. The composition or the method of claim 1, wherein the composition is applied to an edible substrate to form an edible coating and the edible coating has a surface friction of less than or equal to 70 grams of force, as measured by a TX XT Texture Analyzer slip rig.

13. The composition or the method of claim 1, wherein the composition is applied to an edible substrate to form an edible coating and the edible coating starts to peel or bubble from the substrate in less than or equal to 80 seconds in a bath of deionized water at 37° C.

14. The composition or the method of claim 1, wherein the composition is applied to an edible substrate to form an edible coating and the edible coating starts to peel or bubble from the substrate in less than or equal to 60 seconds in a bath of deionized water at 37° C.

15. The composition or the method of claim 1, wherein the composition is applied to an edible substrate to a weight gain of about 1% to about 3% of the edible substrate.

16. The composition or the method of claim 1, wherein the composition is sprayed onto a substrate and the spray time is less than 13.6 minutes under the spray conditions defined by Table 1.

17. The composition or the method of claim 1, wherein the composition is sprayed onto a substrate and the spray time is less than 12.8 minutes under the spray conditions defined by Table 1.

18. The composition or the method of claim 1, wherein the composition is sprayed onto a substrate and the spray time is at least 6% more efficient compared to an otherwise identical edible coating composition not comprising the lubricant.

19. The composition or the method of claim 1, wherein the edible substrate is a nutritional supplement, a pharmaceutical, a tablet, a capsule, a softgel, a granule, a microparticle, a nanoparticle, a seed, a sugar sphere, gum chew base, chewable gel, or a gummy substrate.

20. The composition or the method of claim 1, wherein the lubricant is a non-allergen oil and/or a non-genetically modified organism (non-GMO) oil.

21. A nutritional supplement, a pharmaceutical, a tablet, a capsule, a softgel, a granule, a microparticle, a nanoparticle, a seed, a sugar sphere, gum chew base, chewable gel, or a gummy substrate comprising an edible substrate and the composition of claim 1.
